(19) United States
(12) Patent Application Publication
(10) Pub. No.: US 2024/0050025 A1
(43) Pub. Date: Feb. 15, 2024

(54) DETECTION AND TREATMENT OF DERMATOLOGICAL CONDITIONS

(71) Applicants: Pablo Prieto, Barcelona (ES); Meda Pacurar, Cluj (RO); Lorand Dobai, Brasov (RO); Mihai-Alexandru Badila, Sibiu (RO); Pedro Luis Gonzales, Barcelona (ES); Carlos Gallardo, Barcelona (ES); Josep Malvehy, Barcelona (ES); Martha Jessenia Padilla Raza, Barcelona (ES)

(72) Inventors: Pablo Prieto, Barcelona (ES); Meda Pacurar, Cluj (RO); Lorand Dobai, Brasov (RO); Mihai-Alexandru Badila, Sibiu (RO); Pedro Luis Gonzales, Barcelona (ES); Carlos Gallardo, Barcelona (ES); Josep Malvehy, Barcelona (ES); Martha Jessenia Padilla Raza, Barcelona (ES)

(21) Appl. No.: 17/766,251

(22) PCT Filed: Oct. 2, 2020
(86) PCT No.: PCT/EP2020/077761

§ 371 Date: Apr. 4, 2022

Related U.S. Application Data
(60) Provisional application No. 62/910,158, filed on Oct. 3, 2019.

(51) Int. Cl. A61B 5/00; G06T 7/00; G06V 10/25

(52) U.S. Cl. CPC A61B 5/441; A61B 5/7267; A61B 5/0077; G06T 7/0012; G06V 10/25; G06T 2207/20081; G06T 2207/20084; G06T 2207/30088; G06T 2207/30201

(57) ABSTRACT

Systems and methods for diagnosing dermatological conditions and determining deviations from normal appearances are provided. In one aspect, a computer-implemented method involves receiving, by an image sensor of a computing device, a plurality of images of a subject and determining, by the computing device, a region of interest for each image in the plurality of images. The computer-implemented method also involves generating, by a neural network disposed within the computing device, a dermatological condition metric related to a severity of at least one dermatological condition exhibited by the region of interest for each image in the plurality of images. The computer-implemented method also involves comparing, at the computing device, the dermatological condition metric to one or more stored metrics and providing, by the computing device and based on the comparison, a titration recommendation.

### CROSS-REFERENCE TO RELATED APPLICATIONS

This patent application claims priority to U.S. Provisional Patent Application No. 62/910,158, filed on Oct. 3, 2019, entitled “Detection and Treatment of Dermatological Conditions”, the contents of which are entirely incorporated herein by reference for all purposes.

### BACKGROUND

The outward appearance of a human face relies on a delicate balance between hormones, oil, and bacteria. Aberrations to this balance can cause the human face to exhibit one or more dermatological conditions, such as acne, inflammation, hives, and so on. While these dermatological conditions can be alleviated by way of products or specialized treatments, identifying dermatological conditions and determining an appropriate product/treatment for a given dermatological condition can be time consuming, logistically difficult, or prohibitively expensive, often involving multiple in-person visits to dermatologists or other skin care professionals.

### SUMMARY

The present disclosure defines systems and methods to quickly diagnose dermatological conditions and determine the optimal products, treatments, or solutions to alleviate the dermatological conditions. Through the combination of neural networks, progression modules, and distortion adjustments, the embodiments herein provide a technical solution that may quickly pinpoint regions of interest on a human face and diagnose deviations from normal appearances.

Accordingly, in a first example embodiment, a method may include receiving, by an image sensor of a computing device, a plurality of images of a subject and determining, by the computing device, a region of interest for each image in the plurality of images. The method also include generating, by a neural network disposed within the computing device, a dermatological condition metric related to a severity of at least one dermatological condition exhibited in the region of interest for each image in the plurality of images. The method also include comparing, at the computing device, the dermatological condition metric to one or more stored metrics and providing, by the computing device and based on the comparison, a titration recommendation.

In a second example embodiment, an article of manufacture may include a non-transitory computer-readable medium, having stored thereon program instructions that, upon execution by a computing system, cause the computing system to perform operations in accordance with the first example embodiment.

In a third example embodiment, a computing system may include at least one processor, as well as memory and program instructions. The program instructions may be stored in the memory, and upon execution by the at least one processor, cause the computing system to perform operations in accordance with the first example embodiment.

In a fourth example embodiment, a system may include various means for carrying out each of the operations of the first example embodiment.

In a fifth example embodiment, a method may include receiving, by an image sensor of a computing device, a plurality of images of a subject. The method may further include receiving, by the computing device, at least one current dermatological treatment plan used by the subject during a current time period. The method may also include generating, by a neural network disposed within the computing device, a dermatological condition metric related to a severity of at least one dermatological condition exhibited by the subject in the plurality of images. The method may yet further include providing, to a trained machine learning (ML) model, the at least one dermatological treatment plan and the dermatological condition metric, where the trained ML model is trained to receive current dermatological treatment plans and dermatological condition metrics for subjects and predict dermatological treatment plans for the subjects during future time periods. The method may additionally include obtaining, from the trained ML, a dermatological treatment plan for the subject during a future time period. The method may also include providing the dermatological treatment plan.

In a sixth example embodiment, an article of manufacture may include a non-transitory computer-readable medium, having stored thereon program instructions that, upon execution by a computing system, cause the computing system to perform operations in accordance with the fifth example embodiment.

In a seventh example embodiment, a computing system may include at least one processor, as well as memory and program instructions. The program instructions may be stored in the memory, and upon execution by the at least one processor, cause the computing system to perform operations in accordance with the fifth example embodiment.

In an eighth example embodiment, a system may include various means for carrying out each of the operations of the fifth example embodiment.

These as well as other embodiments, aspects, advantages, and alternatives will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 is a block diagram of a method for treating dermatological conditions, in accordance with an example embodiment.

FIG. 2 depicts facial regions of input images, in accordance with an example embodiment.

FIG. 3 depicts a spatial relationship between a device and a subject, in accordance with an example embodiment.

FIG. 4A depicts a neural network, in accordance with an example embodiment.

FIG. 4B illustrates a convolutional block of a neural network, in accordance with example embodiments.

FIG. 5 depicts comparative images of a subject before and after dermatological treatment, in accordance with an example embodiment.

FIG. 6 is a flowchart of a method, in accordance with an example embodiment.

FIG. 7 is a flowchart of a method, in accordance with an example embodiment.

FIG. 8A is a flowchart of a method, in accordance with an example embodiment.

FIG. 8B is a flowchart of a method, in accordance with an example embodiment.

FIG. 9 depicts a schematic drawing of a computing device, in accordance with an example embodiment.

FIG. 10 depicts a schematic drawing of a server device cluster, in accordance with an example embodiment.

FIG. 11 depicts phases of a machine learning system, in accordance with an example embodiment.

FIG. 12 is a flowchart of a method, in accordance with an example embodiment.

FIG. 13 is a flowchart of a method, in accordance with an example embodiment.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein. Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

### I. Techniques and Methods for Treating Dermatological Conditions

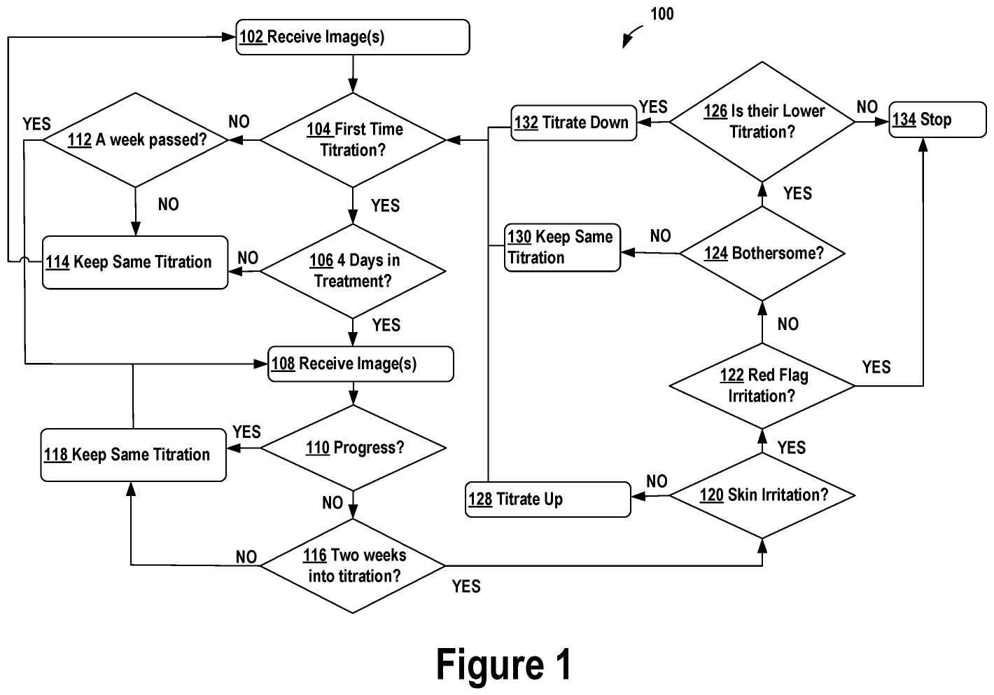

FIG. 1 is a block diagram of method 100 for treating dermatological conditions, in accordance with an example embodiment. Method 100 receives input images and may proceed through one or more steps to generate predictions and make recommendations regarding the input images. In particular, method 100 may generate one or more dermatological recommendations based on predicted dermatological conditions in the input images. Method 100 may be carried out by a computing device, such as computing device 900 described below.

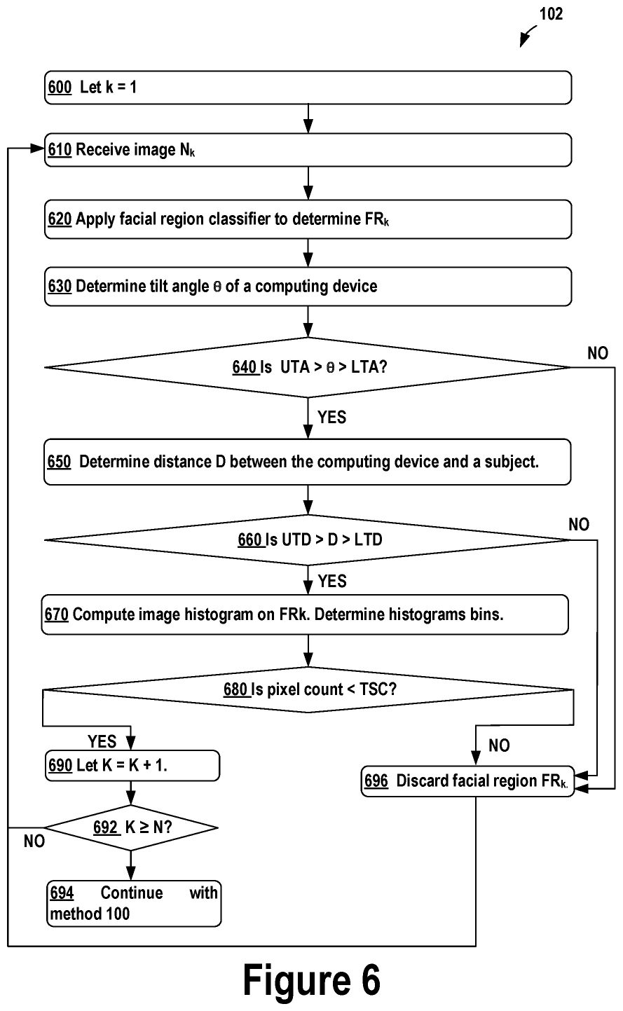

Method 100 begins at block 102, where the computing device receives N input images related to a given subject. Once the N input images are received, the computing device can determine N facial regions FRₖ for the N input images, where k ranges from 1 to N. A facial region for an input image may indicate a region of the image that represents a face, such as a human face, of the given subject. In some examples, the facial region may have a square or rectangular shape. In other examples, the facial region can have a different shape than a square or rectangle; e.g., an oval or elliptical shape, a triangular shape, a hexagonal shape, etc. Detailed procedures related to block 102 are described below in the context of FIG. 6.

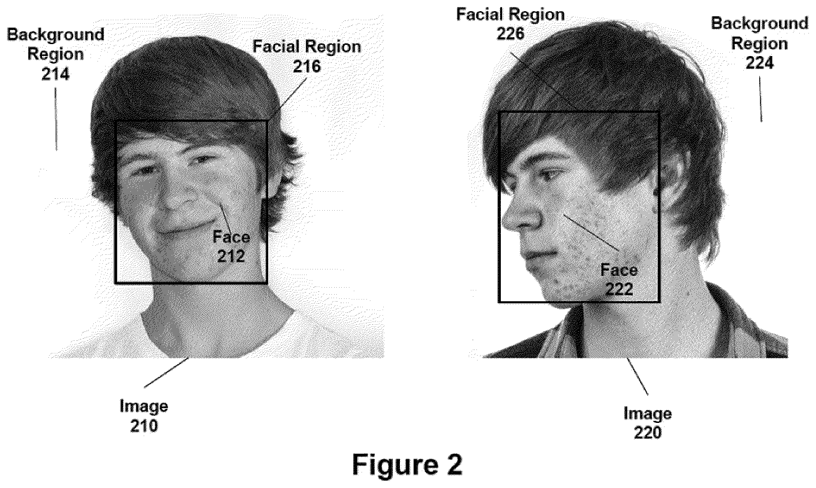

As an example related to block 102, FIG. 2 shows an example frontal input image 210 that includes face 212 in front of background region 214. FIG. 2 shows that face 212 is partially surrounded by facial region 216, which takes the form a box shape. FIG. 2 also shows an example profile input image 220 that includes face 222 in front of background region 224. FIG. 2 shows that face 222 is partially surrounded by facial region 226. Both facial region 216 and facial region 226 are examples of facial regions determined at block 102.

In some embodiments, during the execution of block 102, the computing device determines, for each facial region FRₖ, 1≤k≤N of the N facial regions, potential distortions associated with facial region FRₖ. Distortions may result from a tilt of the computing device, the distance of the computing device from the subject, or an interfering external light signal. Other distortions are also possible. In some examples and in response to determining potential distortions, the computing device may request a replacement image. In other examples, the computing device may perform one or more face-related transformations on facial region FRₖ to correct the distortions. Detailed procedures for adjusting potential distortions are described below in the context of FIG. 6.

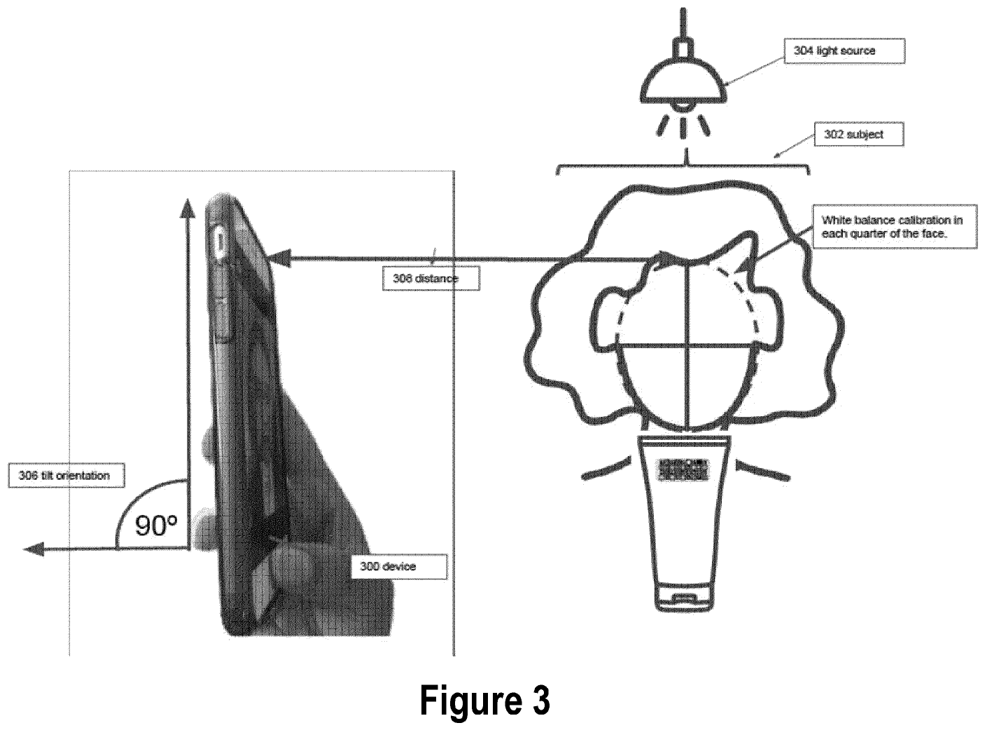

As an example of a distortions, FIG. 3 depicts a spatial relationship between device 300 and subject 302. Device 300 may capture one or more input images (e.g., input images N) of subject 302. Notably, while device 300 is depicted as a mobile computing device, device 300 may take on other forms of computing devices.

FIG. 3 shows that device 300 may be titled at angle θ with respect to the tilt orientation 306. That is, device 300 may be rotated 0 degrees away from tilt orientation 306. Depending on the value of angle θ, the extent of the facial region of subject 302 that can be captured by device 300 may be limited. Tilt orientation 306 is illustrated as aligned with a vertical axis; however, other alignments are also possible. In examples, device 300 may determine a measurement of tilt angle θ through internal sensors, such as embedded gyroscopes, accelerometers, and so on.

FIG. 3 also shows that device 300 is located at distance 308 from subject 302. Depending on the value of distance 308, the extent of the facial region of subject 302 that can be captured by device 300 may be limited. In examples, device 300 may determine a value of distance 308 by comparing the size of a facial region captured on subject 302 to a threshold metric.

FIG. 3 further shows that device 300 may receive a light signal 310 from light source 304. Depending on the magnitude of light signal 310, the ability for device 300 to accurately discriminate the facial nuances of subject 302 may be limited. More particularly, light signal 310 may inadvertently increase the brightness of images captured by device 300. In examples, device 300 may determine a magnitude of light signal 310 by comparing tonal histograms of images captured by device 300.

At block 104, the computing device determines whether a current titration amount assigned to the subject is a first titration amount assigned to the subject. In examples, the current titration amount could relate to a chemical concentration of a particular dermatological product provided to the subject. The determining could include, for example, querying a database, such as a database in server cluster 1000, to determine whether the subject had previous titrations. If the computing device determines that the current titration amount is the first titration amount assigned to the subject, then the computing device proceeds to block 106. Otherwise, the computing device determines that the current titration amount is not the first titration amount assigned to the subject and proceeds to block 112.

At block 106, the computing device determines whether the subject has had at least 4 days of treatment using the current titration amount. The determining could include, for example, querying a database, such as a database in server cluster 1000, to determine whether the subject has had at least 4 days of treatment using the current titration amount. If the computing device determines that the subject has had at least 4 days of treatment using the current titration amount, then the computing device proceeds to block 108. Otherwise, the computing device determines that the subject has not had at least 4 days of treatment using the current titration amount and proceeds to block 114.

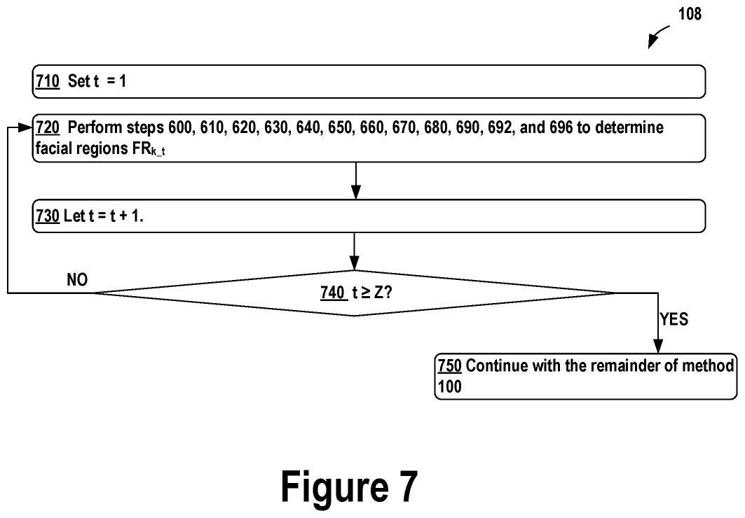

At block 108, the computing device receives N input images and determines facial regions FR k for the N input images. In example embodiments, block 108 may involve receiving N images of over the course of multiple time periods (e.g., receiving N input images every week over the course of two weeks, receiving N images every week over the course of three weeks, etc.). Images captured during different time periods may then be compared to determine whether the subject has exhibited any progress in alleviating a dermatological condition. Detailed procedures related to block 108 are described below in the context of FIG. 7.

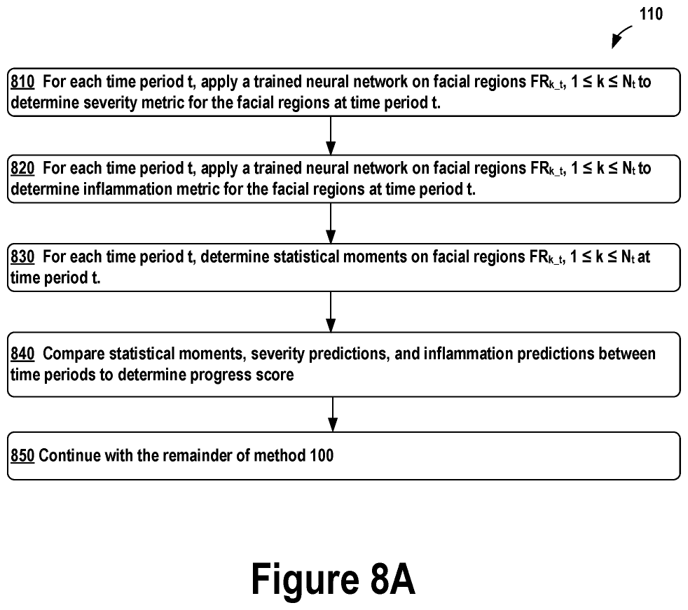

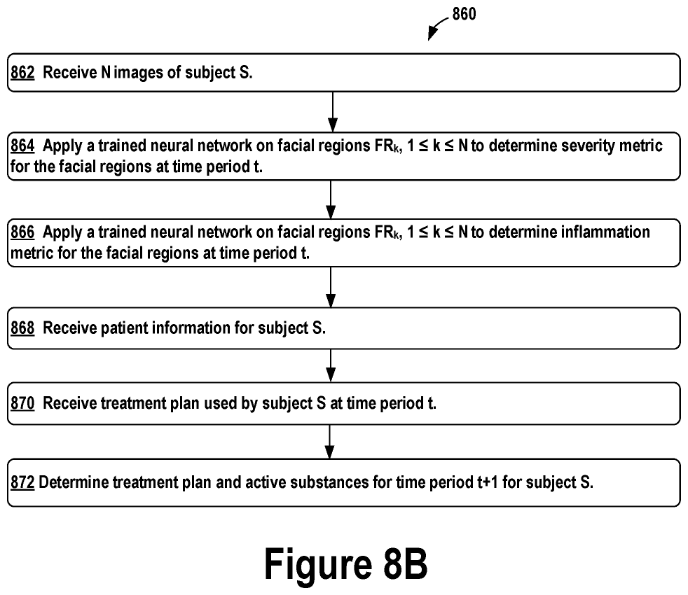

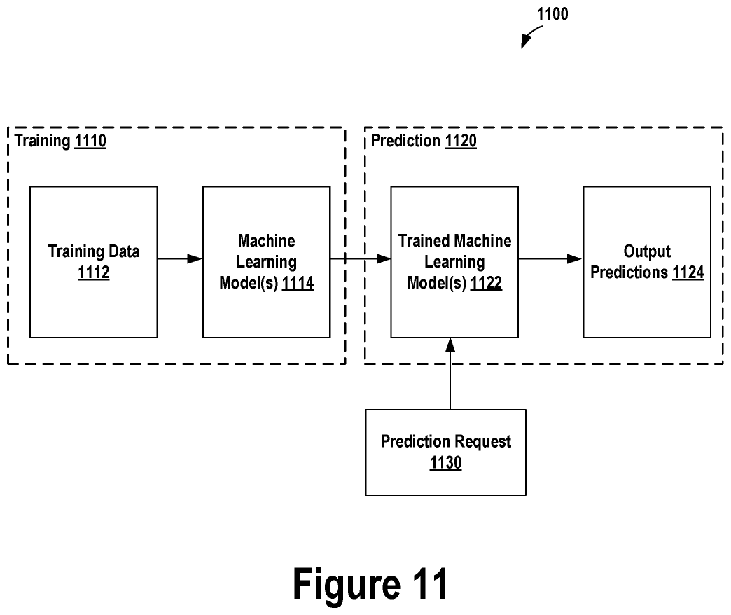

At block 110, the computing device determines whether the subject has exhibited any progress in alleviating a dermatological condition. This could entail, for example, applying, for the facial regions determined in block 108, one or more trained neural networks to predict dermatological conditions. The neural network(s) of block 110 may be an example of a trained machine learning system as discussed below in the context of FIG. 11. During training, the neural network(s) of block 110 may be trained with input images of known dermatological conditions. Then, the trained version of the neural network(s) may be used to make inferences/predictions with respect to each of the facial regions. These inferences/predictions may include acne severity metrics and/or inflammation metrics. Notably, if the computing device determines that the subject has exhibited progress in alleviating the dermatological condition, then the computing device proceeds to block 118. Otherwise, the computing device determines that the subject has not exhibited progress in alleviating the dermatological condition and proceeds to block 116. Detailed procedures related to block 110 are described below in the context of FIG. 8A.

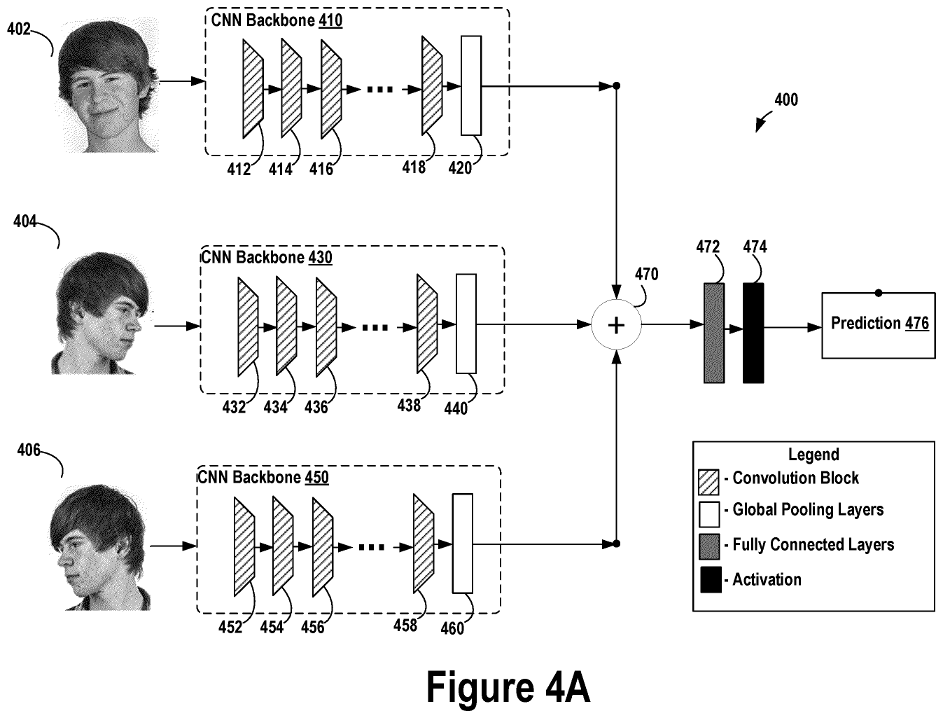

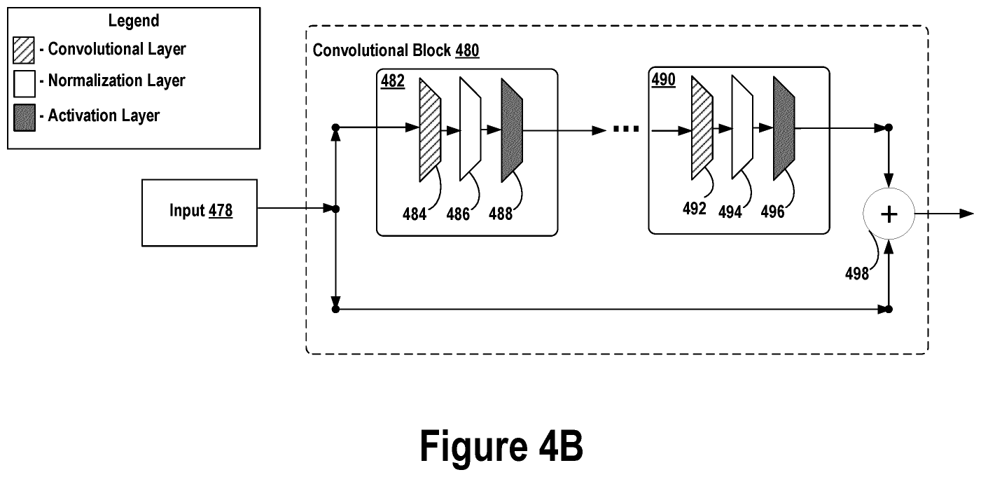

As an example related to block 110, FIG. 4A depicts neural network 400, in accordance with an example embodiment. Neural network 400 may be a convolutional neural network used to implement the procedures of block 110, for example. As shown in FIG. 4A, neural network 400 may perform convolution, activation, pooling, or inference tasks using a combination of convolution layers, activation layers, pooling layers, and fully connected layers.

To conceptually illustrate these layers, a legend is shown at lower right portion of FIG. 4A. The legend indicates that convolutional blocks of neural network 400 are shown filled with diagonal lines, global pooling layers are shown in white, fully connected layers are shown in grey, and activation layers are shown in black. In some examples, part or all of neural network 400 may be implemented using specialized hardware and/or software designed for neural networks; e.g., specialized neural network circuitry, software libraries with software supporting neural networks.

Neural network 400 is shown to include three CNN backbones: CNN backbone 410, CNN backbone 430, and CNN backbone 450. When making an inference, each CNN backbone may be applied on a different one of N facial regions FRₖ. For example, CNN backbone 410 may be applied on frontal region 402, CNN backbone 430 may be applied on right side region 404, and CNN backbone 450 may be applied on left side region 406. In some embodiments, the dimensions of frontal region 402, right side region 404, and left side region 406 may be equal. For example, the dimensions of frontal region 402, right side region 404, and left side region 406 may be 224×224×3.

Each CNN backbone may contain one or more interconnected convolutional blocks. For example, CNN backbone 410 includes convolutional blocks 412, 414, 416, and 418, CNN backbone 430 includes convolutional blocks 432, 434, 436, and 438, and CNN backbone 450 includes convolutional blocks 452, 454, 456, and 458. The number of convolutional blocks in a given CNN backbone may vary (e.g., as indicated by the ellipsis). In examples, the number of convolutional blocks in a given CNN backbone may depend on the size of the facial regions provided into neural network 400 (e.g., the size of frontal region 402, right side region 404, and left side region 406) or the number of training examples available to neural network 400, among other possibilities.

FIG. 4B illustrates convolutional block 480 that neural network 400 can use to perform convolutional block operations, in accordance with example embodiments. For example, convolutional block 480 can be used to implement some or all of convolutional blocks 412, 414, 416, 418, 432, 434, 436, 438, 452, 454, 456, and 458.

Convolutional block 480 may include one or more inter-connected sub-blocks. For example, convolutional block 480 includes sub-block 482, which receives input 478, and sub-block 490, which receives the output from sub-block 482. Each sub-block in convolutional block 480 may include a convolutional layer, a normalization layer, and an activation layer. For example sub-block 482 includes convolutional layer 484, normalization layer 486, and activation layer 488 and sub-block 490 includes convolutional layer 492, normalization layer 494, and activation layer 496.

Generally speaking, a convolution layer includes one or more filters used to filter respective inputs. Each filter works over a subset of an input image or volume. For example, if an input to a convolution layer represents an image of X*Y pixels having Z different color inputs (e.g., an image of size X*Y pixels in either Z=1 for a greyscale color image, Z=3 for a red-green-blue (RGB) image, or Z=4 for a cyan-magenta-yellow-black (CMYK) image), then the input to the convolution layer may be considered to be a X*Y*Z volume of input pixels*colors. Then, a filter of the convolution layer filters a portion of the X*Y*Z volume (e.g., filter X1*Y1*Z1 pixels/colors, where X1≤X, Y1≤Y, and Z1=Z). In another example, suppose an input to convolutional layer was a 100×100 pixel image in CMYK format (Z=4). As such, the convolution layer receives the 100×100×4 volume of pixels/colors as an input volume and acts to convolve a 3×3×4 filter over the 100×100×4 volume. To convolve the filter over the volume, the convolution layer slides the filter across the width and height of the input volume and compute dot products between the entries of the filter and the input at each position that the filter is on the input volume. As the convolution layer slides the filter over the width and height of the input volume, the convolution layer generates a 2-dimensional activation map that gives the responses of that filter at every spatial position of the input volume.

In examples, the convolutional layers of convolutional block 480 may use 3×3×3 filters with stride 3 to reduce the spatial dimensions (e.g., height and width) of an input volume and may use Z number of 1×1×3 filters with stride 1 to reduce (e.g., Z<depth of input volume) or expand (e.g., Z>depth of input volume) the depth of an input volume.

Once the activation map is created, a normalization layer can be applied to adjust the activations in the activation map. Generally speaking, normalization acts to reduce the variance of the values in the activation map. In examples, the normalization layers within the sub-blocks of convolutional block 480 may include a batch normalization layer.

After normalization, an activation function may be applied to determine which values of the activation map are to be provided to a subsequent layer. For example, suppose the activation map has values in the range [0, 1] and that only activation map values above 0.4 are to be provided to a subsequent layer. Then, an activation function could map activation map values in the range [0, 0.4] to 0 (representing no activation), and map activation map values in the range (0.4, 1] to 1 (representing activation). More generally, the activation function can determine whether the output of the convolution layer (e.g., the activation map) is to be provided to a subsequent layer. In examples, the activation layers within the sub-blocks of convolutional block 480 may use sigmoid/logistic activation functions, a hyperbolic tangent activation functions, or a rectified linear unit (ReLU) functions.

The number of sub-blocks in convolutional block 480 may vary (e.g., as indicated by the ellipsis). In examples, the number of sub-blocks in convolutional block 480 may depend on the size of the facial regions provided into neural network 400 (e.g., the size of frontal region 402, right side region 404, and left side region 406) or the number of training examples available to neural network 400, among other possibilities.

Adder 498 of convolutional block 480 may operate as a skip connection for input 478. That is, adder 498 may concatenate or sum the output from the last sub-block of convolutional block 480 (in this example sub-block 490) with input 478. In some cases, adder 498 may operate to add a portion/sub-slice of input 478 to the output from the last sub-block of convolutional block 480.

Returning to FIG. 4A, each CNN backbone is shown to include a global pooling layer. For example, CNN backbone 410 includes global pooling layer 420, CNN backbone 430 includes global pooling layer 440, and CNN backbone 450 includes global pooling layer 460. Each global pooling layer may execute a global pooling operation to average of the channels of the last convolutional block of their respective CNN backbone. For example, global pooling layer 420 may average the channels of convolutional block 418, global pooling layer 440 may average the channels of convolutional block 438, and global pooling layer 460 may average the channels of convolutional block 458. The output from global pooling layer 420, global pooling layer 440, and global pooling layer 460 may then be passed to concatenator 470.

As a particular example of a global pooling operation, if the output dimensions for a last convolutional block is 7×7×160, a global pooling layer may average across the 7×7 channels values to create a 1×1×160 dimension volume (e.g., a 160 length vector).

In some embodiments, CNN backbone 410, CNN backbone 430, and CNN backbone 450 may be identical structures. That is, the weights for CNN backbone 410, CNN backbone 430, and CNN backbone 450 may be the same. As another way of putting this, layers 412, 432, and 452 may have equivalent weights, layers 414, 434, and 454 may have equivalent weights, and so on.

In some embodiments, CNN backbone 410, CNN backbone 420, and CNN backbone 450 may take the form of three separate structures. In some embodiments, CNN backbone 410, CNN backbone 420, and CNN backbone 450 may take the form of a single structure. That is, during training, a single CNN backbone may trained, and then at inference time, the single trained CNN backbone may be applied three times: once on frontal region 402, once on right side region 404, and once on left side region 406. In such a scenario, the operations of concatenator 470 may be put on hold until the single trained CNN backbone has processed frontal region 402, right side region 404, and left side region 406.

Concatenator 470 may operate to concatenate the output from CNN backbone 410, CNN backbone 430, and CNN backbone 450. For example, after global pooling layer 420, global pooling layer 440, and global pooling layer 460 each respectively pass a vector to concatenator 470, concatenator 470 may concatenate these vectors. More specifically, if the vectors passed by global pooling layer 420, global pooling layer 440, and global pooling layer 460 each have a length of 640, concatenator 470 may concatenate these vectors and output a vector of length 1920. The output of concatenator 470 may be passed to fully connected layer 472.

In some embodiments, neural network 400 includes activation layer 474. Activation layer 474 may receive, as input, the output from fully connected layer 472 and may output prediction 476, which may take the form of a D dimensional vector, where D is the number of possible predictions that could be made about a dermatological condition of the subject associated with frontal region 402, right side region 404, and left side region 406. For example, D could be 2 (e.g., a binary prediction distribution), 5, or 10. Each output in the D dimensional vector may represent the probability of a certain class (i.e., possible prediction). For example, a D dimensional vector could appear as [0.1, 0.4, 0.5], where the probability of class 1 is 10%, the probability of class 2 is 40%, and the of class 3 is 50%.

In other embodiments, neural network 400 may skip activation layer 474 and may directly use the output from fully connected layer 472 to predict a continuous value (e.g., based on a regression) about a dermatological condition of the subject associated with frontal region 402, right side region 404, and left side region 406.

It should be noted that the architecture of neural network 400 is not intended to be limiting with respect to example embodiments or techniques described herein. In other embodiments, neural network 400 may have a different architecture with more, fewer, and/or different layers.

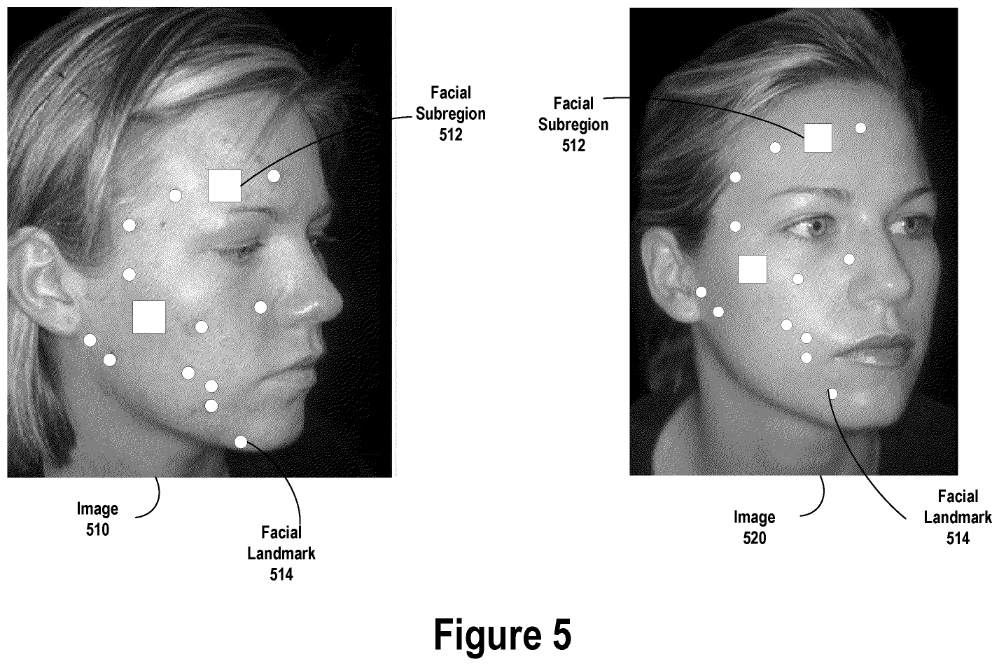

As another example related to block 110, FIG. 5 depicts comparative images of a subject before and after dermatological treatment, in accordance with an example embodiment. In particular, image 510 may represent an image of a subject before dermatological treatment, whereas image 520 may represent an image of the same subject after dermatological treatment has started. Image 510 is shown to contain facial landmarks, shown in FIG. 5 as white circles and include facial landmark 514 near the bottom of before image 510, and facial sub-regions, shown in FIG. 5 as white squares and include facial sub-region near the middle-center of before image 510. The facial landmarks and facial sub-regions may represent areas with dermatological abnormalities, such as excessive redness or swelling. These facial landmarks and facial sub-regions also appear on image 520, except that the dermatological abnormalities are shown to have subsided. Accordingly, by comparing facial landmarks and facial sub-regions of image 510 to image 520, the computing device may determine whether the subject has exhibited any progress in alleviating a dermatological condition.

At block 112, the computing device determines whether at least a week has passed with the subject using the current titration amount. The determining could include, for example, querying a database, such as a database in server cluster 1000, to determine whether the subject has been using the current titration amount for at least a week. If the computing device determines that at least a week has passed with the subject using the current titration amount, then the computing device proceeds to block 108. Otherwise, the computing device determines that at least a week has not passed with the subject using the current titration amount and proceeds to block 114.

At block 114, the computing device determines that the current titration amount should remain the same. After block 114, method 100 may continue back to block 102.

At block 116, the computing device determines whether at least two weeks have passed with the subject using the current titration amount. The determining could include, for example, querying a database, such as a database in server cluster 1000, to determine whether the subject has been using the current titration amount for at least two weeks. If the computing device determines that at least two weeks have passed with the subject using the current titration amount, then the computing device proceeds to block 120. Otherwise, the computing device determines that at least two weeks have not passed with the subject using the current titration amount and proceeds to block 118.

At block 118, the computing device determines that the current titration amount should remain the same. After block 118, method 100 may continue back to block 108.

At block 120, the computing device determines whether the subject exhibits any irritation. In some embodiments, the determining could include applying, on the facial regions determined in block 108, one or more trained neural networks to predict whether the irritation is present. The one or more trained neural networks may be an example of a trained machine learning system as discussed below in the context of FIG. 11. During training, the one or more trained neural networks of block 120 may be trained with input images exhibiting irritation and input images not exhibiting irritation. After training, the trained version of the one or more trained neural network may be an example of trained machine learning model(s) 1122. In some embodiments, the computing device can determine whether irritation is present using the procedures of block 110. For example, if the acne severity metric/inflammation metric determined at block 110 are above a threshold metric, then the computing device may determine that the subject is exhibiting irritation. In some embodiments, the determining could include receiving from a user interface of the computing device, such as input/output unit 908, an indication of whether the skin is irritated. Notably, if the computing device determines that irritation is present on the subject, then the computing device proceeds to block 122. Otherwise, the computing device determines that irritation is not present on the subject, and proceeds to block 128.

At block 122, the computing device determines whether the subject exhibits any red flag irritation. A red flag irritation may refer to a severe irritation that needs to be immediately attended to. In some embodiments, determining a red flag irritation includes applying, for each facial region determined in block 108, one or more trained neural networks to predict whether a red flag irritation is present. The one or more trained neural networks may be an example of a trained machine learning system as discussed below in the context of FIG. 11. During training, the one or more trained neural networks of block 122 may be trained with input images exhibiting red flag irritation and input images not exhibiting red flag irritation. After training, the trained version of the one or more trained neural network of block 122 may be an example of trained machine learning model(s) 1122 If the computing device determines that a red flag irritation is present, then the computing device proceeds to block 134. Otherwise, the computing device determines that a red flag irritation is not present and proceeds to block 124.

At block 124, the computing device determines whether the skin irritation is bothersome for the subject. For example, the skin irritation may be causing unpleasant side effects for the subject, including skin peeling or itchiness, among other possibilities. The determining could include, for example, receiving from a user interface of the computing device, such as input /output unit 908, an indication of whether the skin irritation is bothersome. If the computing device determines that the skin irritation is bothersome, then the computing device proceeds to block 126. Otherwise, the computing device determines that the skin irritation is not bothersome and proceeds to block 130.

At block 126, the computing device determines whether a lower titration amount exists. The determining could include, for example, querying a database, such as a database in server cluster 1000, to determine whether a lower titration amount to the current titration amount exists. If the computing device determines that a lower titration amount exists, then the computing device proceeds to block 132. Otherwise, the computing device determines that a lower titration amount does not exist and proceeds to block 134.

At block 128, the computing devices increases the current titration amount to a higher titration amount. For example, a current titration amount at level 8 may be increased to level 9 or level 10. After block 128, method 100 may continue back to block 104.

At block 130, the computing device determines the current titration amount should remain the same. After block 130, method 100 may continue back to block 104. Notably, block 130, block 118, and block 114 may represent the same logical step and may be shown as separate blocks in method 100 for the purposes of clarity.

At block 132, the computing devices lowers the current titration amount to a lower titration amount. For example, a current titration amount at level 8 may be lowered to level 7 or level 6. After block 132, method 100 may continue back to block 104.

At block 134, method 100 may determine that the dermatological conditions on the subject have subsided and thus that the dermatological treatment was successful. Method 100 may then terminate.

FIG. 6 is a flowchart of a method for the procedures of block 102 of method 100, in accordance with an example embodiment. For example, the computing device performing method 100 may perform at least some of the procedures of blocks 600, 610, 620, 630, 640, 650, 660, 670, 680, 690, 692, 694, and 696 while performing the procedures of block 102 of method 100. The method of FIG. 6 may be simplified by the removal of any one or more of the blocks shown therein. Further, blocks of the method of FIG. 6 may be performed in parallel (e.g., blocks 630-640, blocks 650-660, and block 670-680 may be carried out in parallel).

At block 600, the computing device lets k=1, where k ranges from 1 to N.

At block 610, the computing device receive image Nₖ. In line with the discussion above, image Nₖ may be a profile image or frontal face image of a subject, such as subject 302. Image Nₖ may be received from a camera component physically attached to the computing device

At block 620, the computing device applies a facial region classifier to determine facial region FRₖ for image Nₖ. Notably, the facial region classifier of block 620 may be an example of a machine learning system as discussed below in the context of FIG. 11. During training, the facial region classifier of block 620 may be trained with input images containing facial regions and input images not containing facial regions. After training, the trained version of the facial region classifier may be an example of trained machine learning model(s) 1122. Then, the trained version of the facial region classifier may be used to make inferences/predictions with respect to image Nₖ. In particular, the trained facial region classifier may be configured to make inferences/predictions by way of Haar Features and/or local binary patterns learned during training.

At block 630, the computing device determines tilt angle θ. Tilt angle θ may be the tilt orientation of the computing device at the time facial region FRₖ was captured. In examples, the computing device may use embedded gyroscopes, accelerometers, or other types of sensors to determine tilt angle θ.

At block 640, the computing device determines whether the measurement of tilt angle θ is smaller than an upper threshold angle UTA and greater than a lower threshold angle LTA. If the computing device determines that the measurement of tilt angle θ is greater than LTA and smaller than UTA, then the computing device proceeds to block 650. Otherwise, the computing device determines that the measurement tilt angle θ is outside the range of [LTA, UTA], and proceeds to block 696.

At block 650, the computing device determines distance D. Distance D may be the distance between the computing device and the subject at the time facial region FRₖ was captured. To determine distance D, the computing device may compute the area of facial region FRₖ and compare the computed area to the areas stored facial regions captured at known distances. For example, the computing device may determine that the area of facial region FR k is 100×100 pixels, and then find N other facial regions (e.g., N=1, 2, 3, 4) that have areas of 100×100 pixels. Then, the computing device may average the distances of the N other facial regions to determine distance D. Other techniques for determining distance D are also possible.

At block 660, the computing device determines whether the measurement of distance D is smaller than an upper threshold distance UTD and greater than a lower threshold distance LTD. If the computing device determines that the measurement of distance D is greater than LTD and smaller than UTD, then the computing device proceeds to block 670. Otherwise, the computing device determines that the measurement of distance D is outside the range of [LTD, UTD], and proceeds to block 696.

At block 670, the computing device computes an image histogram of facial region FRₖ, which may include a tonal distribution of pixels that constitute facial region FRₖ. The horizontal axis of this image histogram may represent tonal variations, with the left side of the horizontal axis representing black tonal spectrum, the middle of the horizontal axis representing grey tonal spectrum, and the right side of the horizontal axis representing white tonal spectrum. The vertical axis of this image histogram may represent that number of pixels with a particular tone.

At block 680, the computing device determines whether the pixel count in one or more histogram bins exceeds threshold pixel count TPC. In some cases, the one or more histogram bins may include histogram bins of the white tonal spectrum. In some cases, the one or more histogram bins may include histogram bins of the black tonal spectrum. If the computing device determines that the pixel count in one or more histogram bins exceeds threshold pixel count TPC, the computing device may proceed to block 696. Otherwise, the computing device proceeds to block 690.

At block 690, the computing device lets k=k+1.

At block 692, the computing device determines whether K≥N. If K≥N, then the computing device proceeds to block 694 and continues with the remainder of method 100; i.e., complete the procedures of block 102 of method 100 and continue method 100 by beginning performance of the procedures of block 104 of method 100. Otherwise, if K<N, then the computing device proceeds to block 610.

At block 696, the computing device discards facial region FRₖ. By discarding face region FRₖ, the computing device effectively will no longer process FRₖ. Then, the computing device may request that a replacement facial region FRₖ be determined, perhaps by returning to block 610.

FIG. 7 is a flowchart of a method for the procedures of block 108 of method 100, in accordance with an example embodiment. For example, the computing device performing method 100 may perform at least some of the procedures of blocks 710, 720, 730, 740, and 750 while performing the procedures of block 108 of method 100.

At block 710, the computing device sets time period t=1. As noted above, since the computing device can receive images of over the course of multiple time periods (e.g., receive N input images every week over the course of two weeks), block 710 may represent an initialization of a first time period of a plurality of time periods Z.

At block 720, the computing device performs steps 600, 610, 620, 630, 640, 660, 670, 680, 690, 692, and 696 as described in FIG. 6 to determine facial regions FRₖ t (where 1≤k≤Nₜ, Nₜ representing the number of images captured at time period t). Put differently, block 720 allows the computing device to capture the dermatological conditions of the subject at time period t.

At block 730, the computing device lets t=t+1. This block may represent the end of the duration of time period t and the beginning of the duration of subsequent time period t+1.

At block 740, the computing device determines whether t≥Z. That is, the computing device determines whether the time period t is the last time period of the plurality of time periods Z. For example, if at block 108 the computing device is configured capture N images every 4 weeks, then Z may equal 4 and every time period t may represent a week. As another example, if at block 108 the computing device is configured capture N images every 5 days, then Z may equal 5 and every time period t may represent a day. Other examples of time periods are also possible.

If K Z, then the computing device proceeds to block 750 and continues with the remainder of method 100; i.e., complete the procedures of block 108 of method 100 and continue method 100 by beginning performance of the procedures of block 110 of method 100. Otherwise, if K<N, then the computing device proceeds back to block 720.

FIG. 8A is a flowchart of a method for the procedures of block 110 of method 100, in accordance with an example embodiment. For example, the computing device performing method 100 may perform at least some of the procedures of blocks 810, 820, 830, 840, and 850 while performing the procedures of block 110 of method 100.

At block 810, the computing device applies, for each time period t, a trained neural network on the facial regions FRₖ t (where 1≤k≤Nₜ) determined in block 108 to determine a single acne severity metric, which may indicate the extent of acne present on the Nₜ facial regions at time period t. The trained neural network may take the form of neural network 400 and may be trained with input images containing facial regions of various levels of acne severity. In examples, the single acne severity metric may include a classification of clear, mild, moderate, or severe acne. In some embodiments, applying the trained neural network on facial regions FR_{k_t} involves applying a CNN backbone to each facial region FR_{k_t} and then combining the results from each CNN backbone to form the single severity metric for time period t, as previously described with respect to FIGS. 4A and 4B. In some embodiments, the single severity metric determined at block 810 may be stored in a database for later use.

At block 820, the computing device applies, for each time period t, a trained neural network on the facial regions FR_{k_t} (where 1≤k≤Nₜ) determined in block 108 to determine a single inflammation metric, which may indicate the extent of inflammation present on the Nₜ facial regions at time period t. The trained neural network may take the form of neural network 400 and may be trained with input images containing facial regions of various levels of inflammation. In examples, the single inflammation metric may include a binary classification (e.g., whether inflammation present or not). In some embodiments, applying the trained neural network to facial regions FR_{k_t} involves applying a CNN backbone to each facial region FR_{k_t} and then combining the results from each CNN backbone to form the single inflammation metric for time period t, as previously described with respect to FIGS. 4A and 4B. In some embodiments, the single inflammation metric determined at block 820 may be stored in a database for later use.

At block 830, the computing device determines, for each time period t, statistical moments for facial regions FR^{k_t} (where 1≤k≤Nₜ). To do this, the computing device may first determine an optimal color space to analyze red tones on facial regions FR_{k_t} by isolating the “a” channel from the CIE L*a*b color space (or H channel in HSV color space) on facial regions FR_{k_t}. Then, the computing device may identify the facial landmarks and sub-regions by comparing tonal deviations on facial regions FR_{k_t}. For example, a facial landmark or a facial sub-region may be identified as an area of on facial regions FR_{k_t} that exhibits a deviation (e.g., 1 standard deviation, 2 standard deviations) from an average tonal distribution of on facial region FR_{k_t}. The computing device may then compute statistical moments with respect to the identified facial landmarks and facial sub-regions. For example, the statistical moments may include a mean tone for all the identified facial landmarks and facial sub-regions, a median tone for all the identified facial landmarks and facial sub-regions, or a variance for all the identified facial landmarks and facial sub-region. Other statistical moments are also contemplated. In some embodiments, the statistical moments determined at block 830 may be stored in a database for later use.

Accordingly, at block 840, the computing device compares the statistical moments /inflammation metrics/severity metrics of the subject between time periods to determine whether the subject has made progress in the dermatological treatment. For example, if the inflammation metrics/severity metrics/statistical moments for a current/recent time period are lower than the inflammation metrics/severity metrics/statistical moments for one or more prior time periods, then progress may be considered achieved.

At block 850, the computing device proceeds with the remainder of method 100; i.e., complete the procedures of block 110 of method 100 and continue method 100 by beginning performance of the procedures of block 116 or 118 of method 100, depending on the results of block 840.

### II. Techniques and Methods for Customized Treatment of Dermatological Conditions

FIG. 8B is a flowchart of a method for determining customized treatments of dermatological conditions, according to example embodiments. Method 860 may involve receiving input images of a given subject S at time period t (e.g., a current time period) and then, using the input images and other factors, generating one or more dermatological products and treatment plans for subject S to employ at time period t+1 (e.g., a future time period). Method 860 may be carried out by a computing device, such as computing device 900 described below.

Method 860 begins at block 862, where the computing device receives N input images related to a given subject S. Once the N input images are received, the computing device can determine N facial regions FRₖ for the N input images, where k ranges from 1 to N. Example procedures for block 862 are described above in the context of FIG. 6.

At block 864, the computing device applies a trained neural network on the facial regions FRₖ t (where 1≤k≤Nₜ) to determine a single acne severity metric, which may indicate the extent of acne present on the N facial regions at time period t. The trained neural network may take the form of neural network 400 and may be trained with input images containing facial regions of various levels of acne severity. In some examples, the single acne severity metric may include a classification of clear, mild, moderate, or severe acne. In other examples, the single acne severity metric may be a numerical value within a certain numerical range (e.g., an integer within the range 0 to 12). Other ways of representing the single acne severity metric are also possible. In some embodiments, applying the trained neural network on facial regions FRₖ involves applying a CNN backbone to each facial region FRₖ and then combining the results from each CNN backbone to form the single severity metric for time period t, as previously described with respect to FIGS. 4A and 4B.

At block 866, the computing device applies a trained neural network on the facial regions FRₖ (where 1≤k≤Nₜ) to determine a single inflammation metric, which may indicate the extent of inflammation present on the N facial regions at time period t. The trained neural network may take the form of neural network 400 and may be trained with input images containing facial regions of various levels of inflammation. In some examples, the single inflammation metric may include a binary classification (e.g., whether inflammation present or not). In other examples, the single inflammation metric may be a numerical value within a certain numerical range (e.g., an integer within the range 0 to 12). Other ways of representing the single inflammation metric are also possible. In some embodiments, applying the trained neural network to facial regions FRₖ involves applying a CNN backbone to each facial region FRₖ and then combining the results from each CNN backbone to form the single inflammation metric for time period t, as previously described with respect to FIGS. 4A and 4B.

At block 868, the computing device receives patient information for subject S. The patient information could include, for example, demographic information about subject S (e.g., Caucasian, Asian, Black, etc.), the age of subject S, or the skin type of subject S (e.g., “normal,” “oily”, or “dry”), among other possibilities. In some implementations, the patient information could be entered into the computing device by subject S via a graphical interface disposed on the computing device. In other implementations, the patient information could be communicatively transmitted to the computing device by a second computing device. For instance, the second computing device may be a remote server device that stores profiles related to various subjects, including subject S. Accordingly, upon request from the computing device, the remote server device could look up a stored profile related to subject S and then transmit information from that stored profile to the computing device. Other ways of receiving the patient information also exist.

At block 870, the computing device receives a treatment plan used by subject S during time period t. The treatment plan may include, for example, the dermatological products used by subject S during time period t, titration amounts of those dermatological products, application instructions for those dermatological products (e.g., frequency of application, quantity of a dermatological product that should be applied, etc.), or the active substances in those dermatological products, among other possibilities. In some implementations, block 868 may involve receiving the treatment plans used by subject S during time period t as well as the previous X time periods (e.g., X=1, 3, 5, 10). Similar to the patient information, the treatment plan used by subject S during time period t could be entered into the computing device via a graphical interface or could be communicatively transmitted to the computing device from a second computing device.

At block 872, the computing device may provide the results from blocks 862, 864, 866, 868, and 870 (e.g., the single acne severity, the single inflammation metric, the patient information, and the treatment plan) to a dermatological machine learning (ML) model. Such a dermatological ML model may take the form of neural network 400, a recurrent neural network (RNN), a decision tree, or any of the machine learning models(s) 1114 described below. Regardless of the form, the dermatological ML model may be configured to receive the results from blocks 862, 864, 866, 868, and 870 as an N-dimensional vector. For example, the results from blocks 862, 864, 866, 868, and 870 may be passed to the dermatological ML model as a plurality of values X1-XN (i.e., X1, X2, X3, X4, X5, X6, X7, X8, X9, and X10 through XN) in N-dimensional vector space. The dermatological ML model may be trained to predict one or more dermatological products and treatment plans for subject S for a future time period (e.g., time period t+1) based on the N-dimensional vector. The prediction made by the dermatological ML model could be communicated to subject S via the aforementioned graphical user interface and/or could be transmitted to a remote server device for storage.

Note that the blocks represented method 860 are used for the purpose of example and are not intended to be limiting with respect to the embodiments herein. Method 860 may be highly configurable and may include more blocks, fewer blocks, or different blocks than those depicted in FIG. 8B. In some cases, one or more blocks may be customized, or may otherwise deviate from the exemplary description above.

### III. Example Computing Devices and Cloud-Based Computing Environments

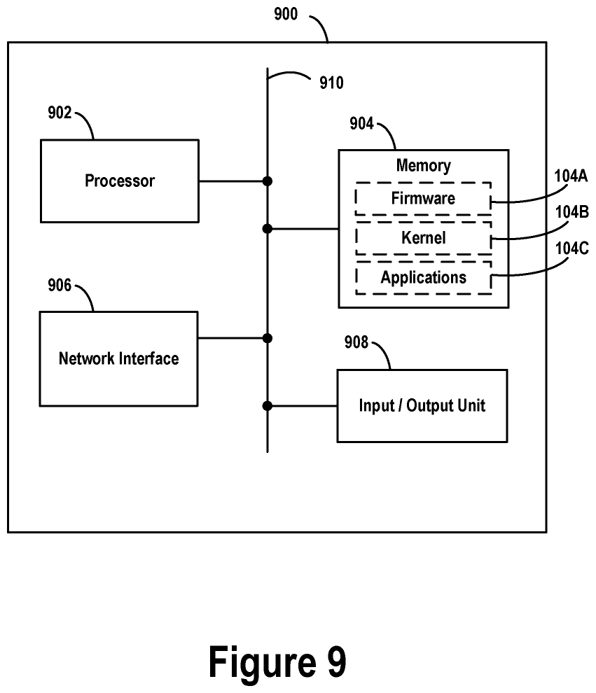

FIG. 900 is a simplified block diagram exemplifying a computing device 900, illustrating some of the components that could be included in a computing device arranged to operate in accordance with the embodiments herein. Computing device 900 could be a client device (e.g., a device actively operated by a user), a server device (e.g., a device that provides computational services to client devices), or some other type of computational platform. Some server devices may operate as client devices from time to time in order to perform particular operations, and some client devices may incorporate server features. In particular, computing device 900 shown in FIG. 9 may be configured to perform at least one function of and/or related to method 100 and neural network 400.

In this example, computing device 900 includes processor 902, memory 904, network interface 906, and an input/output unit 908, all of which may be coupled by a system bus 910 or a similar mechanism. In some embodiments, computing device 900 may include other components and/or peripheral devices (e.g., detachable storage, printers, and so on).

Processor 902 may be one or more of any type of computer processing element, such as a central processing unit (CPU), a co-processor (e.g., a mathematics, graphics, or encryption co-processor), a digital signal processor (DSP), a network processor, and/or a form of integrated circuit or controller that performs processor operations. In some cases, processor 902 may be one or more single-core processors. In other cases, processor 902 may be one or more multi-core processors with multiple independent processing units. Processor 902 may also include register memory for temporarily storing instructions being executed and related data, as well as cache memory for temporarily storing recently-used instructions and data.

Memory 904 may be any form of computer-usable memory, including but not limited to random access memory (RAM), read-only memory (ROM), and non-volatile memory (e.g., flash memory, hard disk drives, solid state drives, compact discs (CDs), digital video discs (DVDs), and/or tape storage). Thus, memory 904 represents both main memory units, as well as long-term storage. Other types of memory may include biological memory.

Memory 904 may store program instructions and/or data on which program instructions may operate. By way of example, memory 904 may store these program instructions on a non-transitory, computer-readable medium, such that the instructions are executable by processor 902 to carry out any of the methods, processes, or operations disclosed in this specification or the accompanying drawings.

As shown in FIG. 9, memory 904 may include firmware 904A, kernel 904B, and/or applications 904C. Firmware 904A may be program code used to boot or otherwise initiate some or all of computing device 900. Kernel 904B may be an operating system, including modules for memory management, scheduling and management of processes, input/output, and communication. Kernel 904B may also include device drivers that allow the operating system to communicate with the hardware modules (e.g., memory units, networking interfaces, ports, and busses), of computing device 900. Applications 904C may be one or more user-space software programs, such as web browsers or email clients, as well as any software libraries used by these programs. Memory 904 may also store data used by these and other programs and applications.

Network interface 906 may take the form of one or more wireline interfaces, such as Ethernet (e.g., Fast Ethernet, Gigabit Ethernet, and so on). Network interface 906 may also support communication over one or more non-Ethernet media, such as coaxial cables or power lines, or over wide-area media, such as Synchronous Optical Networking (SONET) or digital subscriber line (DSL) technologies. Network interface 906 may additionally take the form of one or more wireless interfaces, such as IEEE 802.11 (Wifi), BLUETOOTH®, global positioning system (GPS), or a wide-area wireless interface. However, other forms of physical layer interfaces and other types of standard or proprietary communication protocols may be used over network interface 906. Furthermore, network interface 906 may comprise multiple physical interfaces. For instance, some embodiments of computing device 900 may include Ethernet, BLUETOOTH®, and Wifi interfaces.

Input/output unit 908 may facilitate user and peripheral device interaction with computing device 900. Input/output unit 908 may include one or more types of input devices, such as a keyboard, a mouse, a touch screen, and so on. Similarly, input/output unit 908 may include one or more types of output devices, such as a screen, monitor, printer, and/or one or more light emitting diodes (LEDs). Additionally or alternatively, computing device 900 may communicate with other devices using a universal serial bus (USB) or high-definition multimedia interface (HDMI) port interface, for example.

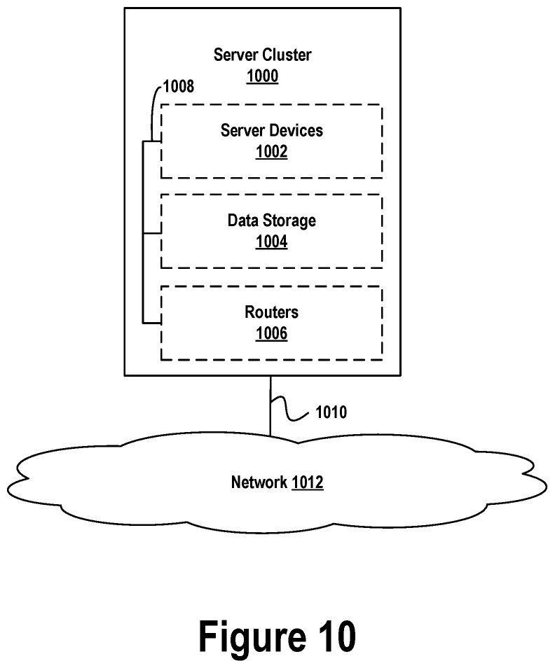

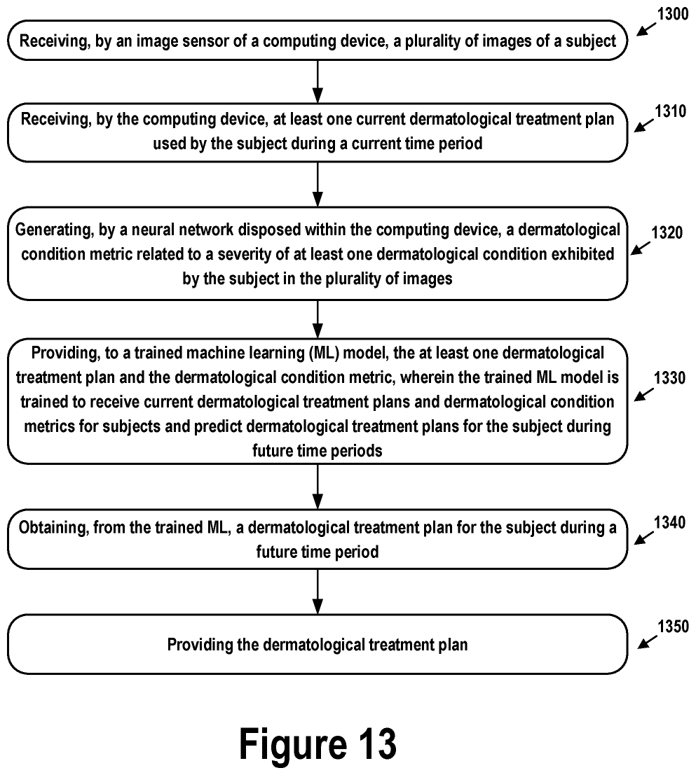

FIG. 10 depicts a cloud-based server cluster 1000 in accordance with example embodiments. In FIG. 10, operations of a computing device (e.g., computing device 900) may be distributed between server devices 1002, data storage 1004, and routers 1006, all of which may be connected by local cluster network 1008. The number of server devices 1002, data storages 1004, and routers 1006 in server cluster 1000 may depend on the computing task(s) and/or applications assigned to server cluster 1000. In particular, the cloud-based server cluster 1000 shown in FIG. 13 may be configured to perform at least one function of and/or related to method 100, neural network 400, and computing device 900.

In examples, server devices 1002 may be configured to perform various computing tasks of computing device 900. Thus, computing tasks can be distributed among one or more of server devices 1002. To the extent that these computing tasks can be performed in parallel, such a distribution of tasks may reduce the total time to complete these tasks and return a result. For purpose of simplicity, both server cluster 1000 and individual server devices 1002 may be referred to as a “server device.” This nomenclature should be understood to imply that one or more distinct server devices, data storage devices, and cluster routers may be involved in server device operations.

Data storage 1004 may be data storage arrays that include drive array controllers configured to manage read and write access to groups of hard disk drives and/or solid state drives. The drive array controllers, alone or in conjunction with server devices 1002, may also be configured to manage backup or redundant copies of the data stored in data storage 1004 to protect against drive failures or other types of failures that prevent one or more of server devices 1002 from accessing units of data storage 1004. Other types of memory aside from drives may be used.

Routers 1006 may include networking equipment configured to provide internal and external communications for server cluster 1000. For example, routers 1006 may include one or more packet-switching and/or routing devices (including switches and/or gateways) configured to provide (i) network communications between server devices 1002 and data storage 1004 via local cluster network 1008, and/or (ii) network communications between the server cluster 1000 and other devices via communication link 1010 to network 1012.

Additionally, the configuration of routers 1006 may be based at least in part on the data communication requirements of server devices 1002 and data storage 1004, the latency and throughput of the local cluster network 1008, the latency, throughput, and cost of communication link 1010, and/or other factors that may contribute to the cost, speed, fault-tolerance, resiliency, efficiency and/or other design goals of the system architecture.

As a possible example, data storage 1004 may include any form of database, such as a structured query language (SQL) database. Various types of data structures may store the information in such a database, including but not limited to tables, arrays, lists, trees, and tuples. Furthermore, any databases in data storage 1004 may be monolithic or distributed across multiple physical devices.

Server devices 1002 may be configured to transmit data to and receive data from data storage 1004. This transmission and retrieval may take the form of SQL queries or other types of database queries, and the output of such queries, respectively. Additional text, images, video, and/or audio may be included as well.

FIG. 11 illustrates a machine learning system 1100, which includes training phase 1110 and prediction phase 1120, in accordance with example embodiments. Some machine learning techniques involve training one or more machine learning systems on an input set of training data. The resulting trained machine learning system may be called a trained machine learning model, for short. For example, FIG. 11 shows training phase 1100 where machine learning model(s) 1114 are being trained on training data 1112 to become trained machine learning model(s) 1112. During prediction phase 1120, trained machine learning model(s) 1112 may receive prediction request 1130 and responsively provide output prediction(s) 1124. In some embodiments, trained machine learning models 1122 can be embodied by neural network 400.

Machine learning models(s) 1114 may include, but are not limited to: an artificial neural network (e.g., a herein-described convolutional neural network), a Bayesian network, a hidden Markov model, a Markov decision process, a logistic regression function, a support vector machine, a suitable statistical machine learning algorithm, and/or other machine learning models.

During training phase 1110, machine learning model (s) 1114 may be trained by providing training data 1112 and by using training techniques, such as but not limited to, unsupervised, supervised, semi-supervised, reinforcement learning, transfer learning, and/or incremental learning techniques.

During prediction phase 1120, trained machine learning model(s) 1122 may receive prediction request 1130 and generate corresponding output prediction(s) 1124 about prediction request 1130. Prediction request 1130 may include information/data similar to that of training data 1112, so as to allow for trained machine learning model(s) 1122 to appropriately make predictions.

In some embodiments, training data 1112 and prediction request 1130 may include a collection of images provided by one or more sources. The collection of images may include images of an object, such as a human face, where the images of the human face are taken under different lighting conditions, images of multiple objects, and/or other images. Other types of input data are possible as well.

In some embodiments, output prediction(s) 1124 may include classifications, maps, numerical values, and/or other output data produced by trained machine learning model(s) 1122 operating on prediction request 1130.

In some embodiments, training phase 1110 and prediction phase 1120 may take place on a single device or server cluster. In other examples, training phase 1110 and prediction phase 1120 may take place on separate devices or separate server clusters. For example, after training phase 1110 has been completed on computing device A but before prediction phase 1120 begins, trained machine learning model(s) 1113 may be provided, installed, and/or otherwise prepared for use by computing device B, which may responsively be configured to execute prediction phase 1120. In some cases, training phase 1110 may occur on a cloud server cluster, such as cloud-based server cluster 1000, whereas the prediction phase 1120 may occur on a device, such as device 900.

In some examples, elements of machine learning system 1110 may be executed and/or accelerated using one or more computer processors and/or on-device coprocessors. The on-device coprocessor(s) can include, but are not limited to one or more graphic processing units (GPUs), one or more tensor processing units (TPUs), one or more digital signal processors (DSPs), and/or one or more application specific integrated circuits (ASICs). Such on-device coprocessors can speed up training phase 1110 and/or prediction phase 1120.

### IV. Example Operations

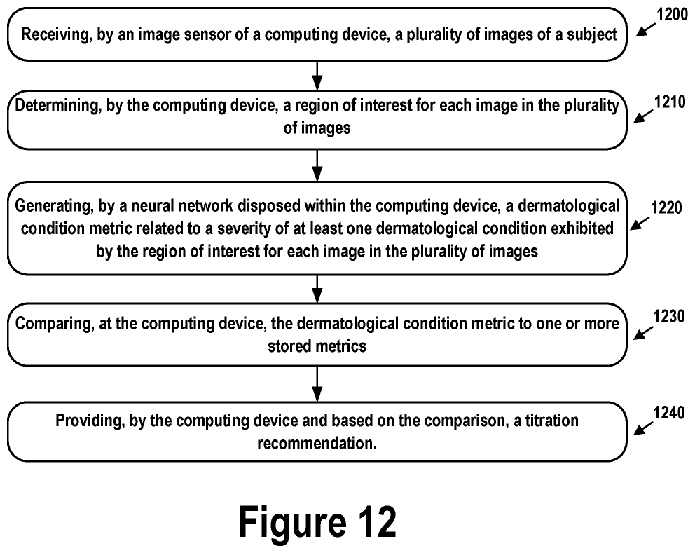

FIG. 12 is a flow chart illustrating an example embodiment. The process illustrated by FIG. 12 may be carried out by a computing device, such as computing device 900, and/or a cluster of computing devices, such as server cluster 1000. However, the process can be carried out by other types of devices or device subsystems. For example, the process could be carried out by a portable computer, such as a laptop or a tablet device.

The embodiments of FIG. 12 may be simplified by the removal of any one or more of the features shown therein. Further, these embodiments may be combined with features, aspects, and/or implementations of any of the previous figures or otherwise described herein.

Block 1200 involves receiving, by an image sensor of a computing device, a plurality of images of a subject

Block 1210 involves determining, by the computing device, a region of interest for each image in the plurality of images.

Block 1220 involves generating, by a neural network disposed within the computing device, a dermatological condition metric related to a severity of at least one dermatological condition exhibited by the region of interest for each image in the plurality of images.

Block 1230 involves comparing, at the computing device, the dermatological condition metric to one or more stored metrics.

Block 1240 involves providing, by the computing device and based on the comparison, a titration recommendation. For example, the providing could include displaying the recommendation on a screen of the computing device, transmitting the recommendation to another computing device, among other possibilities.

In some embodiments generating the dermatological condition metric comprises applying the neural network to the region of interest for each image in the plurality of images.

In some embodiments the neural network comprises a convolutional neural network with separate backbone structures. In these embodiments applying the neural network to the region of interest for each image in the plurality of images comprises: applying, for the region of interest for each image in the plurality of images, one of the separate backbone structures; and combining outputs from the separate backbone structures into a single output.

In some embodiments the neural network comprises a convolutional neural network with a single backbone structure. In these embodiments applying the neural network to the region of interest for each image in the plurality of images comprises: applying, for the region of interest for each image in the plurality of images, the single backbone structure; and combining outputs from the single backbone structure into a single output.

In some embodiments the titration recommendation comprises a recommendation to decrease titration if the comparison is that the dermatological condition metric is greater than the one or more stored metrics.

In some embodiments the titration recommendation comprises a recommendation to keep a same titration if the comparison is that the dermatological condition metric is less than the one or more stored metrics.

Some embodiments further involve determining, at the computing device and based on the region of interest, whether the plurality of images are distorted and if the determination is that the plurality of images are distorted, requesting, by the computing device, a plurality of replacement images.

In some embodiments determining whether the plurality of images are distorted comprises determining whether a tilt orientation of the computing device exceeds a upper tilt threshold or falls below a lower tilt threshold.

In some embodiments determining whether the plurality of images are distorted comprises determining whether a distance between the computing device and the subject exceeds a upper distance threshold or falls below a lower distance threshold.

In some embodiments determining the distance comprises comparing an area of the region of interest to areas of one or more stored regions of interest.

In some embodiments the computing device is configured to compute a tonal distribution histogram of pixels that constitute the region of interest for each image in the plurality of images. In these embodiments determining whether the plurality of images are distorted comprises determining whether a pixel count in one or more histogram bins on the tonal distribution histogram exceeds a threshold pixel count.

In some embodiments the one or more histogram bins may comprise a histogram bin in a white tonal spectrum or a histogram bin in a black tonal spectrum.

In some embodiments the plurality of images comprise at least one profile image of the subject and at least one front facing image of the subject.

In some embodiments the at least one dermatological condition comprises a skin inflammation condition or an acne severity condition.

In some embodiments the computing device is a mobile computing device and the neural network is trained on a remote computing device.

In some embodiments the region of interest is a facial region representing at least a portion of a human face.

FIG. 13 is a flow chart illustrating an example embodiment. The process illustrated by FIG. 13 may be carried out by a computing device, such as computing device 900, and/or a cluster of computing devices, such as server cluster 1000. However, the process can be carried out by other types of devices or device subsystems. For example, the process could be carried out by a portable computer, such as a laptop or a tablet device.

The embodiments of FIG. 13 may be simplified by the removal of any one or more of the features shown therein. Further, these embodiments may be combined with features, aspects, and/or implementations of any of the previous figures or otherwise described herein.

Block 1300 involves receiving, by an image sensor of a computing device, a plurality of images of a subject.

Block 1310 involves receiving, by the computing device, at least one current dermatological treatment plan used by the subject during a current time period.

Block 1320 involves generating, by a neural network disposed within the computing device, a dermatological condition metric related to a severity of at least one dermatological condition exhibited by the subject in the plurality of images.

Block 1330 involves providing, to a trained machine learning (ML) model, the at least one current dermatological treatment plan and the dermatological condition metric, where the trained ML model is trained to receive current dermatological treatment plans and dermatological condition metrics for subjects and predict dermatological treatment plans for the subjects during future time periods.

Block 1340 involves obtaining, from the trained ML, a dermatological treatment plan for the subject during a future time period.

Block 1350 involves providing the dermatological treatment plan.

Some embodiments involve receiving, by the computing device, patient information related to the subject, and providing, to the trained ML model, the patient information. In such embodiments, the trained ML model is trained to receive current dermatological treatment plans, dermatological condition metrics, and patient information for subjects and predict dermatological treatment plans for the subjects during future time periods.

Some embodiments involve receiving, by the computing device, at least one previous dermatological treatment plan used by the subject during a previous time period, and providing, to the trained ML model, the at least one previous dermatological treatment plan. In such embodiments, the trained ML model is trained to receive current dermatological treatment plans, dermatological condition metrics, and previous dermatological treatment plans for subjects and predict dermatological treatment plans for the subjects during future time periods.

### V. Conclusion

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer readable medium such as a storage device including RAM, a disk drive, a solid state drive, or another storage medium.

The computer readable medium can also include non-transitory computer readable media such as computer readable media that store data for short periods of time like register memory and processor cache. The computer readable media can further include non-transitory computer readable media that store program code and/or data for longer periods of time. Thus, the computer readable media may include secondary or persistent long term storage, like ROM, optical or magnetic disks, solid state drives, compact-disc read only memory (CD-ROM), for example. The computer readable media can also be any other volatile or non-volatile storage systems. A computer readable medium can be considered a computer readable storage medium, for example, or a tangible storage device.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. A computer-implemented method comprising:
receiving, by an image sensor of a computing device, a plurality of images of a subject;
determining, by the computing device, a region of interest for each image in the plurality of images;
generating, by a neural network disposed within the computing device, a dermatological condition metric related to a severity of at least one dermatological condition exhibited in the region of interest for each image in the plurality of images;
comparing, at the computing device, the dermatological condition metric to one or more stored metrics; and
based on the comparison, providing, by the computing device, a titration recommendation.

2. The computer-implemented method of claim 1, wherein generating the dermatological condition metric comprises applying the neural network to the region of interest for each image in the plurality of images.

3. The computer-implemented method of claim 1, wherein the neural network comprises a convolutional neural network with separate backbone structures, and wherein applying the neural network to the region of interest for each image in the plurality of images comprises:
applying, for the region of interest for each image in the plurality of images, one of the separate backbone structures; and
combining outputs from the separate backbone structures into a single output.

4. The computer-implemented method of claim 1, wherein the neural network comprises a convolutional neural network with a single backbone structure, and wherein applying the neural network to the region of interest for each image in the plurality of images comprises:
applying, for the region of interest for each image in the plurality of images, the single backbone structure; and
combining outputs from the single backbone structure into a single output.

5. The computer-implemented method of claim 1, wherein the titration recommendation comprises a recommendation to decrease titration if the comparison is that the dermatological condition metric is greater than the one or more stored metrics.

6. The computer-implemented method of claim 1, wherein the titration recommendation comprises a recommendation to keep a same titration if the comparison is that the dermatological condition metric is less than the one or more stored metrics.

7. The computer-implemented method of claim 1, further comprising:
determining, at the computing device and based on the region of interest, whether the plurality of images are distorted; and
if the determination is that the plurality of images are distorted, requesting, by the computing device, a plurality of replacement images.

8. The computer-implemented method of claim 7, wherein determining whether the plurality of images are distorted comprises determining whether a tilt orientation of the computing device exceeds a upper tilt threshold or falls below a lower tilt threshold.

9. The computer-implemented method of claim 7, wherein determining whether the plurality of images are distorted comprises determining whether a distance between the computing device and the subject exceeds a upper distance threshold or falls below a lower distance threshold.

10. The computer-implemented method of claim 9, wherein determining the distance comprises comparing an area of the region of interest to areas of one or more stored regions of interest.

11. The computer-implemented method of claim 7, wherein the computing device is configured to compute a tonal distribution histogram of pixels that constitute the region of interest for each image in the plurality of images, and wherein determining whether the plurality of images are distorted comprises determining whether a pixel count in one or more histogram bins on the tonal distribution histogram exceeds a threshold pixel count.

12. The computer-implemented method of claim 11, wherein the one or more histogram bins may comprise a histogram bin in a white tonal spectrum or a histogram bin in a black tonal spectrum.

13. The computer-implemented method of claim 1, wherein the plurality of images comprise at least one profile image of the subject and at least one front facing image of the subject.

14. The computer-implemented method of claim 1, wherein the at least one dermatological condition comprises a skin inflammation condition or an acne severity condition.

15. The computer-implemented method of claim 1, wherein the computing device is a mobile computing device, and wherein the neural network is trained on a remote computing device.

16. The computer-implemented method of claim 1, wherein the region of interest is a facial region representing at least a portion of a human face.

17. A computing device, comprising:
one or more processors; and
data storage, wherein the data storage has stored thereon computer-executable instructions that, when executed by the one or more processors, cause the computing device to carry out functions comprising the computer-implemented method of any one of claims 1-16.

18. A computer-implemented method comprising:
receiving, by an image sensor of a computing device, a plurality of images of a subject;
receiving, by the computing device, at least one current dermatological treatment plan used by the subject during a current time period;
generating, by a neural network disposed within the computing device, a dermatological condition metric related to a severity of at least one dermatological condition exhibited by the subject in the plurality of images;
providing, to a trained machine learning (ML) model, the at least one current dermatological treatment plan and the dermatological condition metric, wherein the trained ML model is trained to receive current dermatological treatment plans and dermatological condition metrics for subjects and predict dermatological treatment plans for the subjects during future time periods;
obtaining, from the trained ML, a dermatological treatment plan for the subject during a future time period; and
providing the dermatological treatment plan.

19. The computing implemented method of claim 18, further comprising:
receiving, by the computing device, patient information related to the subject, and
providing, to the trained ML model, the patient information, wherein the trained ML model is trained to receive current dermatological treatment plans, dermatological condition metrics, and patient information for subjects and predict dermatological treatment plans for the subjects during future time periods.

20. The computer implemented method of claim 18, further comprising:
receiving, by the computing device, at least one previous dermatological treatment plan used by the subject during a previous time period, and
providing, to the trained ML model, the at least one previous dermatological treatment plan, wherein the trained ML model is trained to receive current dermatological treatment plans, dermatological condition metrics, and previous dermatological treatment plans for subjects and predict dermatological treatment plans for the subjects during future time periods.
